(19) United States
(12) Patent Application Publication
(10) Pub. No.: US 2016/0375271 A1
(43) Pub. Date: Dec. 29, 2016

(54) APPARATUS AND METHODS FOR COMPREHENSIVE ULTRASOUND SKIN TREATMENT

(71) Applicant: SLENDER MEDICAL LTD., Herzliya (IL)

(72) Inventors: Liat TSOREF, Tel Aviv (IL); Gregory KRASNOV, Haifa (IL); Haim AZHARI, Doar-Na Misgav (IL); Yossi GROSS, Moshav Mazor (IL)

(73) Assignee: SLENDER MEDICAL LTD., Herzliya (IL)

(21) Appl. No.: 15/038,846

(22) PCT Filed: Nov. 21, 2014
(86) PCT No.: PCT/IB2014/066237

§ 371 Date: May 24, 2016

Related U.S. Application Data
(60) Provisional application No. 61/908,155, filed on Nov. 24, 2013.
(60) Provisional application No. 61/973,631, filed on Apr. 1, 2014.

(51) Int. Cl. A61N 7/00

(52) U.S. Cl. CPC A61N 7/00; A61N 2007/0034; A61N 2007/0056

(57) ABSTRACT

Cartridge apparatus (230) for use with a handle (220) is provided. The apparatus includes a housing (500) which: (a) includes a handle-coupling portion (235) shaped and sized to securely couple the housing to the handle and to facilitate separation of the housing from the handle, (b) shaped to define a skin-application portion (40) on an outer surface of the housing, and (c) includes at least one ultrasound element (60) in acoustic communication with the skin-application portion and configured to transmit ultrasound energy to the skin through the skin-application portion when the cartridge is attached to the handle. The cartridge apparatus also includes an inflatable element (250) inflated with a fluid, the fluid being in fluid communication with the skin-application portion. Other applications are also described.

### CROSS-REFERENCE TO RELATED APPLICATIONS

The present application claims priority from U.S. Provisional Application 61/973,631 to Tsoref et al., entitled “Apparatus and methods for comprehensive ultrasound skin treatment,” filed Apr. 1, 2014, and U.S. Provisional Application 61/908,155, entitled, “Apparatus and methods for comprehensive ultrasound skin treatment,” filed Nov. 24, 2013, both of which are assigned to the assignee of the present patent application and is incorporated herein by reference.

### FIELD OF THE APPLICATION

Applications of the present invention relate in general to tissue treatment by application of energy thereto, and particularly to methods and apparatus for treatment by application of ultrasound energy to the skin.

### BACKGROUND OF THE APPLICATION

Systems for applying energy to biological tissue are known. Such energy application may be intended to heal injured tissue, ablate tissue, or improve the appearance of tissue.

Skin, the body's largest organ, is composed of multiple layers. The outer layer, epidermis, is divided into several sublayers. The outer most layer of the epidermis is the stratum corneum which mostly consists of dead cells that lack nuclei and organelles. Beneath the epidermis lies the dermis skin layer, which is composed of two layers, the upper papillary layer and the lower reticular layer.

A major structural component of the dermis skin layer is collagen, a fibrous protein, which contributes to skin strength and elasticity. As such, collagen formation, and in contrast, age-related collagen decline, lead to changes in mechanical properties of the skin, such as texture and resilience. Thermal treatment of the skin can lead to thermal shrinkage of collagen, which occurs by the dissociation of heat-sensitive bonds of the collagen molecule (denaturation). Thermal denaturing of collagen typically results in a tightening effect of the skin.

Application of focused ultrasound energy to the skin can lead to thermal contraction and destruction of collagen through both mechanical and biochemical pathways. Collagen remodeling through a wound healing response, occurs over time together with associated neocollagenesis. Collagen remodeling typically results in a desired tightening of the skin.

Visible effects of aging or damage to the skin are disturbing to many individuals and therefore methods for rejuvenation of maturing or damaged skin are of interest. Some skin rejuvenation methods include application of energy to heat selected areas of the skin in order to obtain an improvement in the appearance of the treated skin (e.g., through thermal shrinkage of collagen and/or collagen remodeling, thereby resulting in tightening of the skin).

Exfoliation involves the removal of old, dead skin cells on the skin's outermost surface, and is typically used to maintain healthy skin. Exfoliation is typically performed during facials, microdermabrasion or chemical peels at medical spas. Exfoliation can be achieved through mechanical and/or chemical means. One of the disadvantages of exfoliation is the high price of some of the products and methods used to achieve it.

### SUMMARY OF APPLICATIONS

For some applications, apparatus is provided for use with skin of a subject. The apparatus comprises a housing and a reservoir within the housing. The reservoir contains a fluid, e.g., a cooling fluid, and is shaped to define a curved surface at an outer boundary of the reservoir.

The apparatus additionally comprises an ultrasound element, e.g., a piezoelectric element, which is typically disposed at a respective location with respect to the curved surface, and placed in acoustic communication with the skin. The ultrasound element can be shaped to define a curved ultrasound element or alternatively a flat ultrasound element. For some applications, a lens is coupled to the ultrasound element and configured to focus ultrasound waves. The ultrasound element typically applies ultrasound energy, e.g., high intensity focused ultrasound (HIFU) energy, to the skin.

For some applications, an air barrier is typically disposed between the curved surface and the ultrasound element such that the curved surface is between the air barrier and the reservoir. The air barrier typically allows almost free vibrating of the ultrasound element during application of energy to the skin. The apparatus additionally comprises a skin-application portion and a fluid disposed between the ultrasound element and the skin-application portion that is in fluid communication with the reservoir. Typically, the curved surface is shaped to define one or more slots which provide the fluid communication between the reservoir and the fluid disposed between the ultrasound element and the skin-application portion.

The apparatus is configured to cause motion, e.g., circulation, of the fluid disposed between the ultrasound element and the skin-application portion, to the reservoir. This circulation of the fluid disposed between the ultrasound element and the skin-application portion to the reservoir typically occurs due to a configuration (e.g., a shape) of the apparatus and not due to actively causing circulation of fluid.

For some applications, the ultrasound element is configured to cause motion of the fluid disposed between the ultrasound element and the skin-application portion, to the reservoir, by heating the fluid disposed between the ultrasound element and the skin-application portion.

Motion of cooling fluid within the housing typically provides sufficient cooling of the ultrasound element such that no additional cooling fluid from outside the housing is required.

For some applications, the apparatus comprises an inflatable element coupled to the housing. The inflatable element, e.g., a balloon, is inflated with a fluid, and the fluid is in fluid communication with the ultrasound element and the skin-application portion.

Typically, the skin-application portion comprises a flexible external surface, e.g., a membrane, which contacts the skin when the skin-application portion is placed on skin of the subject. As noted hereinabove, the fluid within the inflatable element is in fluid communication with the ultrasound element and the skin-application portion. The fluid typically applies pressure and outwardly deforms the flexible surface. The fluid typically causes inflation and protruding of the membrane, which results in improved contact between the membrane and the skin.

Typically, a total volume of the fluid in the apparatus is at least 1 cc or less than 50 cc or between 1 and 50 cc. The inflatable element is inflated to a volume of at least 0.5 cc, or less than 50 cc or between 0.5 and 50 cc (e.g., 0.5-5 cc, or 5-50 cc), and the volume of fluid disposed between the ultrasound element and the skin-application portion is typically at least 0.5 cc or less than 10 cc or between 0.5 and 10 cc. Additionally, the inflatable element has a fluid pressure of at least 2 kPa or less than 25 kPa or between 2 and 25 kPa. The volume and pressure of fluid in the inflatable element, typically ensure a fluid volume of 0.5-10 cc between the ultrasound element and the skin-application portion. This volume of 0.5-10 cc generally facilitates inflation and protruding of the membrane, which results in improved contact between the membrane and the skin.

For some applications, a cartridge apparatus for use with a hand-held device, e.g., a handle, is provided. The cartridge typically comprises a housing, the housing: (a) comprising a handle-coupling portion shaped and sized to securely couple the housing to the handle, e.g., by clicking into place, and to facilitate separation of the housing from the handle, (b) shaped to define a skin-application portion on an outer surface of the housing, and (c) comprising at least one ultrasound element in acoustic communication with the skin-application portion and configured to transmit ultrasound energy to the skin through the skin-application portion, when the cartridge is attached to the handle. The cartridge additionally comprises an inflatable element inflated with a fluid, the fluid being in fluid communication with the skin-application portion.

The handle typically comprises electronic circuitry which is activated by a user when the cartridge is attached to the handle and the skin-application portion is positioned in contact with the skin. Activation of the electronic circuitry generates electrical current which activates the ultrasound element to transmit ultrasound energy to the skin through the skin-application portion.

There is therefore provided in accordance with some applications of the present invention, cartridge apparatus for use with a handle, the apparatus including:

a housing, (a) including a handle-coupling portion shaped and sized to securely couple the housing to the handle and to facilitate separation of the housing from the handle, (b) shaped to define a skin-application portion on an outer surface of the housing, and (c) including at least one ultrasound element in acoustic communication with the skin-application portion and configured to transmit ultrasound energy to the skin through the skin-application portion when the cartridge is attached to the handle; and

an inflatable element inflated with a fluid, the fluid being in fluid communication with the skin-application portion.

For some applications, the skin-application portion includes a flexible external surface configured to contact the skin, and the fluid is positioned to apply pressure and outwardly deform the flexible surface.

For some applications, a volume of the fluid in fluid communication with the skin-application portion is 0.5-10 cc.

For some applications, a total volume of the fluid is 1-50 cc.

For some applications, the inflatable element has a fluid pressure of 2-25 kPa.

For some applications, a volume of the inflatable element is 0.5-50 cc.

For some applications, a pressure in the inflatable element inflated with the fluid is 2-25 kPa.

For some applications, the apparatus includes an electrical socket configured to receive electrical current from the handle.

There is further provided in accordance with some applications of the present invention, apparatus, including:

a housing;

a skin-application portion, configured to be placed in contact with skin of a subject;

at least one ultrasound element configured to be placed in acoustic contact with the skin and to apply ultrasound energy to the skin via the skin-application portion; and

an inflatable element coupled to the housing and inflated with a fluid, the fluid being in fluid communication with the ultrasound element and the skin-application portion.

For some applications, the apparatus includes a replaceable cartridge in which the inflatable element is disposed, and the cartridge is removably coupled to the housing.

For some applications, the apparatus includes a replaceable cartridge in which the inflatable element and the at least one ultrasound are disposed, and the cartridge is removably coupled to the housing.

For some applications, the skin-application portion includes a flexible external surface configured to contact the skin, and the fluid applies pressure and outwardly deforms the flexible surface.

For some applications, a volume of the fluid disposed between the ultrasound element and the skin-application portion is 0.5-10 cc.

For some applications, a total volume of the fluid is 1-50 cc. For some applications, the inflatable element has a fluid pressure of 2-25 kPa.

For some applications, the inflatable element is inflated to a volume of 0.5-50 cc.

For some applications, a pressure in the inflatable element inflated with the fluid is 2-25 kPa.

There is further yet provided in accordance with some applications of the present invention, cartridge apparatus, for use with an ultrasound device that includes a housing, a skin-application portion, and at least one ultrasound element, the cartridge apparatus including:

a cartridge configured to be removably attached to the housing;

a fluid port; and

an inflatable element disposed within the cartridge and inflated with a fluid, the fluid port being sized and shaped to provide fluid communication between the fluid in the inflatable element and the skin-application portion, when the cartridge is attached to the housing.

For some applications, the cartridge is shaped to define an outer surface thereof, and the outer surface of the cartridge is not inflatable.

For some applications, the skin-application portion includes a flexible external surface configured to contact the skin, and the fluid applies pressure and outwardly deforms the flexible surface, when the cartridge is attached to the housing.

For some applications, a volume of the fluid disposed between the ultrasound element and the skin-application portion is 0.5-10 cc.

For some applications, a total volume of the fluid in the apparatus is 1-50 cc, when the cartridge is attached to the housing.

For some applications, the inflatable element has a fluid pressure of 2-25 kPa.

For some applications, the inflatable element is inflated to a volume of 0.5-50 cc.

For some applications, a pressure in the inflatable element inflated with the fluid is 2-25 kPa.

For some applications, the cartridge apparatus further includes the at least one ultrasound element.

For some applications, the apparatus includes a coupling mechanism configured to couple the cartridge to the housing and to allow fluid communication between the fluid in the inflatable element and the skin-application portion, when the cartridge is attached to the housing.

For some applications, the cartridge apparatus further includes the at least one ultrasound element.

There is further provided in accordance with some applications of the present invention, apparatus, including:

a skin-application portion, configured to move across skin of a subject;

at least one ultrasound element coupled to the skin-application portion and configured to be placed in acoustic contact with the skin, and configured to apply ultrasound energy to the skin;

circuitry configured to generate a current responsive to motion of the skin-application portion; and

a control unit, which is configured to receive the current, to determine, responsively thereto, whether the skin-application portion has been moving for a predetermined duration of time, with respect to the skin, and to drive the ultrasound element to apply the ultrasound energy to the skin responsively to determining that the skin-application portion has been moving for more than one second with respect to the skin.

For some applications, the predetermined duration of time is at least one second, and the control unit is configured to determine whether the skin-application portion has been moving for at least one second.

For some applications, the predetermined duration of time is at least 0.3 seconds, and the control unit is configured to determine whether the skin-application portion has been moving for at least 0.3 seconds.

For some applications, the predetermined duration of time is 0.3-3 seconds, and the control unit is configured to determine whether the skin-application portion has been moving for 0.3-3 seconds.

There is further provided in accordance with some applications of the present invention, apparatus including:

a skin-application portion, configured to move across a skin surface of a subject;

at least one ultrasound element coupled to the skin-application portion and configured to be placed in acoustic contact with the skin, and configured to apply ultrasound energy to the skin;

one or more optical sensors configured to transmit a signal toward the skin surface and receive a reflection of the signal that reflects off the skin surface;

a control unit, which is configured to receive the signal, and to determine, responsively thereto, whether the skin-application portion is placed on a flat skin surface or a curved skin surface; and

circuitry configured to generate a current responsively to determining that the skin-application portion is placed on the flat skin surface.

For some applications, the apparatus is shaped to define a skin-contact surface plane, and each one of the one or more optical sensors is positioned to transmit energy from the each of the one or more sensors at an angle that is 90 degrees with respect to the skin-contact surface plane.

For some applications, the apparatus is shaped to define a skin-contact surface plane, and each one of the one or more optical sensors is positioned to transmit energy from the each of the one or more sensors at an angle that is greater than 90 degrees with respect to the skin-contact surface plane.

There is further provided in accordance with some applications of the present invention, apparatus, including:

an eyeglasses frame; and

an ultrasound element coupled to the eyeglasses frame and configured to be placed in acoustic contact with skin of an eye bag of a subject, and to apply ultrasound energy to the skin of the eye bag.

There is therefore still provided in accordance with some applications of the present invention, apparatus for use with skin of a subject, including:

a housing;

a reservoir within the housing, containing a fluid, and shaped to define a curved surface at an outer boundary of the reservoir;

an ultrasound element, disposed at a respective location with respect to the curved surface, configured to be placed in acoustic communication with the skin, and configured to apply ultrasound energy to the skin;

an air barrier disposed between the curved surface and the ultrasound element such that the curved surface is between the air barrier and the reservoir;

a skin-application portion; and

a fluid disposed between the ultrasound element and the skin application portion, and in fluid communication with the reservoir.

For some applications, the apparatus is configured to cause motion of the fluid disposed between the ultrasound element and the skin application portion, to the reservoir.

For some applications, the ultrasound element is configured to cause motion of the fluid disposed between the ultrasound element and the skin application portion, to the reservoir, by heating the fluid disposed between the ultrasound element and the skin application portion.

For some applications, the curved surface is shaped to define one or more slots which provide the fluid communication between the reservoir and the fluid disposed between the ultrasound element and the skin application portion.

For some applications, the apparatus includes one or more valves configured to direct the flow of fluid from the reservoir to the fluid disposed between the ultrasound element and the skin application portion.

For some applications, the apparatus includes a pump configured to pump fluid from the fluid reservoir to the fluid disposed between the ultrasound element and the skin application portion.

For some applications, the ultrasound element includes a piezoelectric ultrasound element.

There is further provided in accordance with some applications of the present invention, apparatus, including:

a handle, at a proximal portion of the apparatus;

a skin-application portion, at a distal portion of the apparatus;

at least one ultrasound element configured to apply ultrasound energy to the skin through the skin-application portion; and

an extension portion that extends 3-10 mm from a distal-most tip of the skin-application portion to a distal-most tip of the apparatus.

For some applications, the extension portion extends 4-7 mm from the distal-most tip of the skin-application portion to the distal-most tip of the apparatus.

For some applications, a width of the extension portion is greater than a width of the skin-application portion.

For some applications, the width of the extension portion is at least two times greater than the width of the skin-application portion.

The present invention will be more fully understood from the following detailed description of applications thereof, taken together with the drawings, in which:

### BRIEF DESCRIPTION OF THE DRAWINGS

FIG. 1 is a schematic illustration of apparatus for use with skin of a subject, in accordance with some applications of the present invention;

FIG. 2 is a schematic illustration of apparatus for use with skin of a subject showing components of the apparatus, in accordance with some applications of the present invention;

FIGS. 3A-C are schematic illustrations of components of the apparatus for use with skin of a subject, in accordance with some applications of the present invention;

FIG. 4 is a schematic illustration of a curved surface shaped to define slots for use in the apparatus for use with skin of a subject, in accordance with some applications of the present invention;

FIGS. 5A-F are schematic illustrations of apparatus for skin treatment, as provided by some applications of the present invention;

FIGS. 6A-E are schematic illustrations of components of cartridge apparatus, as provided by some applications of the present invention;

FIG. 7 is a schematic illustration of an electrical socket of the cartridge apparatus, as provided by some applications of the present invention;

FIG. 8 is a schematic illustration of cartridge apparatus coupled to a handle, as provided by some applications of the present invention;

FIG. 9 is a schematic illustration of apparatus for skin treatment comprising a fluid port for providing fluid communication between fluid in the inflatable element and the skin-application portion, in accordance with some applications of the present invention;

FIG. 10 is a flow chart depicting control of ultrasound transmission in the apparatus for skin treatment as provided by some applications of the present invention;

FIG. 11 is a schematic illustration of a treatment site, as provided by some applications of the present invention;

FIGS. 12A-D are schematic illustration of apparatus for skin treatment, in accordance with some applications of the present invention;

FIG. 13 is a schematic illustration of apparatus for treatment of eye bags, as provided by some applications of the present invention; and

FIG. 14 is a schematic illustration of a curved ultrasound element for use with the apparatus for treatment of eye bags of the present invention.

### DETAILED DESCRIPTION OF APPLICATIONS

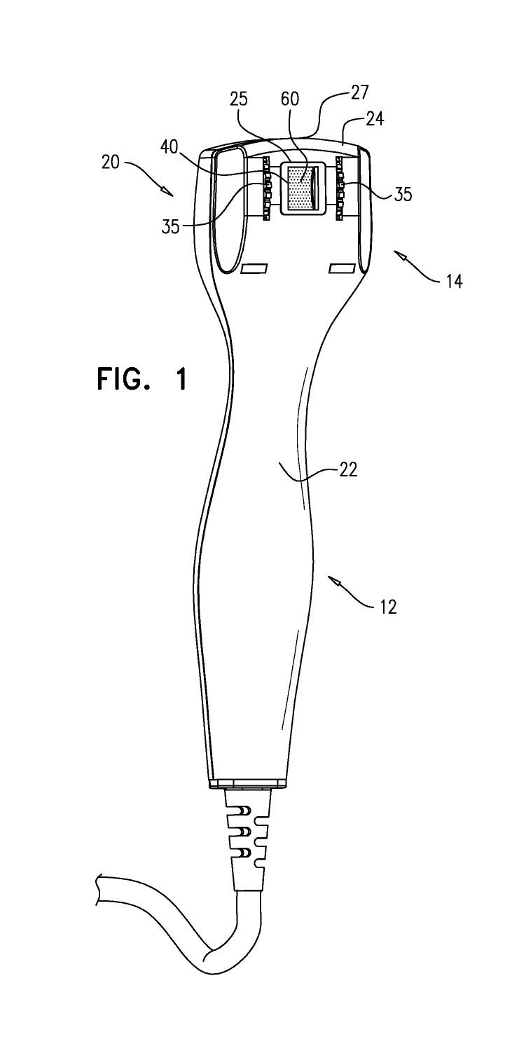

FIG. 1 is a schematic illustration of apparatus 20 for use with skin of a subject, in accordance with some applications of the present invention. Apparatus 20 typically comprises a handheld apparatus comprising a skin-application portion 40 that is moved across a selected region of skin of a subject. Typically, apparatus 20 applies ultrasound energy to the skin for treatment thereof. Application of ultrasound energy to the skin surface with subsequent heating of underlying skin layers typically leads to collagen regeneration and remodeling, resulting in skin tightening and rejuvenation.

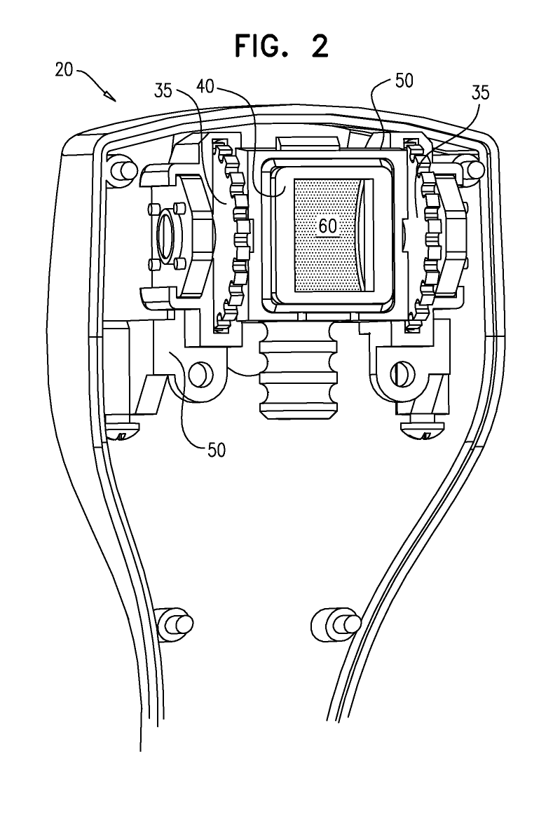

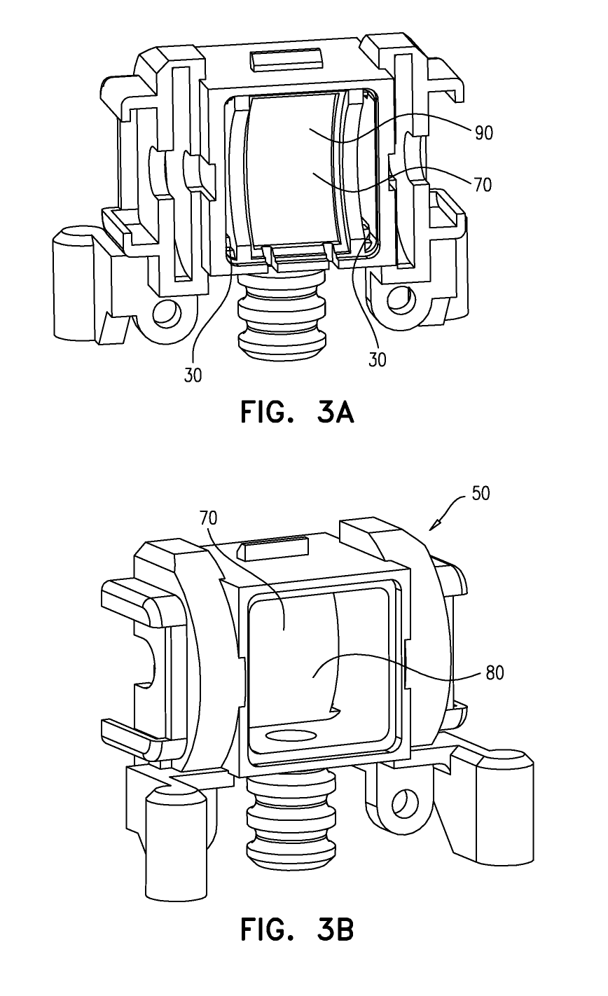

Reference is made to FIGS. 2 and 3A-C, which show components of apparatus 20, in accordance with some applications of the present invention. Apparatus 20 comprises a housing 50 and an ultrasound element 60 which is placed in acoustic communication with the skin, and is configured to apply ultrasound energy, e.g., high intensity focused ultrasound (HIFU) energy, to the skin. Ultrasound element 60 applies ultrasound energy to the skin through skin-application portion 40.

FIG. 3A shows a front view of housing 50, and FIG. 3B shows a back view of housing 50. Typically, a reservoir 80 is present within the housing, containing a fluid, e.g., a cooling fluid, and shaped to define a curved surface 70 at an outer boundary of the reservoir.

FIG. 3C shows a front view of housing 50 having ultrasound element 60 fitted within the housing. Typically but not necessarily, ultrasound element 60 is shaped to define a curved surface disposed at a respective location with respect to curved surface 70, and is typically (but not necessarily) parallel to curved surface 70. An air barrier 90 (FIG. 3A) is disposed between curved surface 70 and the ultrasound element 60, such that curved surface 70 is between the air barrier and fluid reservoir 80. Reference numeral 90 for the air barrier generally indicates the location of the air barrier within housing 50.

The air barrier typically allows vibrating of ultrasound element 60 with relatively low damping during application of HIFU energy to the skin. It is noted that for other applications, e.g., applications in which low energy is applied for monitoring and/or imaging purposes, ultrasound devices typically do not provide an air barrier between the ultrasound element and the surface behind the ultrasound element.

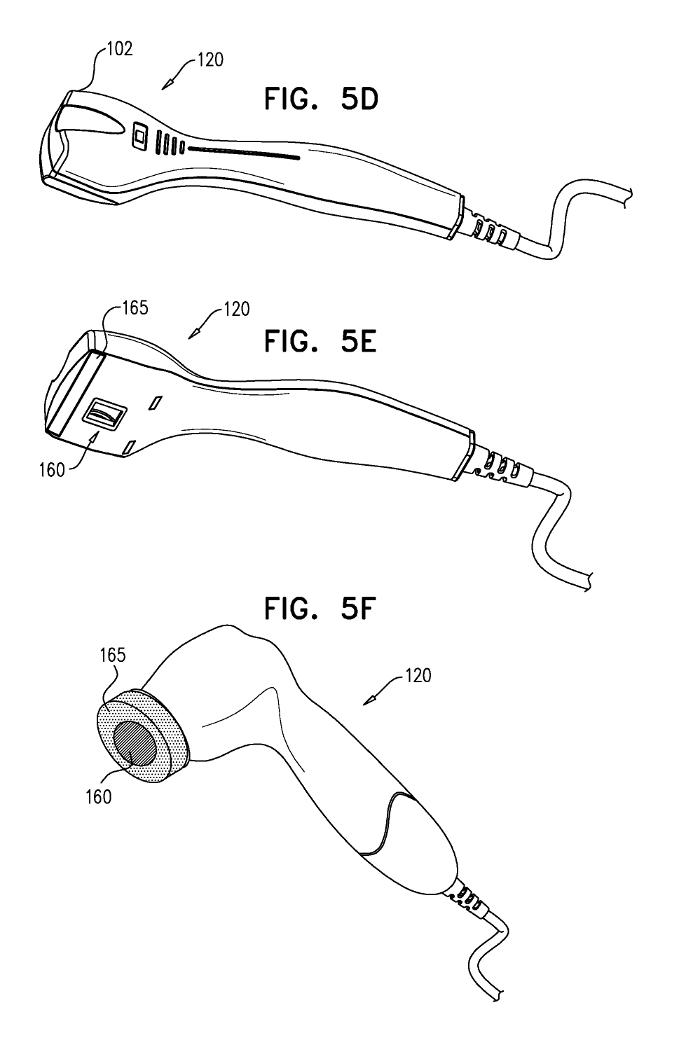

Typically, a fluid, e.g., a cooling fluid (such as 40% Dowtherm SR-1 fluid manufactured by DOW Chemical Company), is disposed between ultrasound element 60 and skin-application portion 40 (the area of fluid between ultrasound element 60 and skin-application portion 40 is shown in an exploded view FIG. 6E, and is indicated by reference numeral 23). The fluid disposed between ultrasound element 60 and skin-application portion 40, is in fluid communication with reservoir 80.

Curved surface 70 is typically shaped to define one or more slots 30 which provide the fluid communication between the fluid in reservoir 80 and the fluid disposed between ultrasound element 60 and skin-application portion 40.

Typically, apparatus 20 is shaped, e.g., by the shape of slots 30, to provide fluid communication and circulation of cooling fluid from the fluid disposed between ultrasound element 60 and skin-application portion 40, to the reservoir. Internal circulation of the cooling fluid within apparatus 20 typically facilitates sufficient heat removal from ultrasound element 60, such that no additional cooling fluid from outside housing 50 is required.

Motion of the fluid disposed between ultrasound element 60 and skin-application portion 40, to reservoir 80, typically occurs due to a configuration (e.g., a shape) of apparatus 20, and not due to actively causing circulation of fluid.

For some applications, ultrasound element 60 causes heating of the fluid disposed between ultrasound element 60 and skin-application portion 40, thereby causing motion of the fluid disposed between the ultrasound element and the skin-application portion, to reservoir 80.

For some applications, apparatus 20 comprises one or more valves (not shown) configured to direct the flow of fluid from reservoir 80 to the fluid disposed between ultrasound element 60 and skin-application portion 40.

Alternatively or additionally, apparatus 20 further comprises a pump configured to pump fluid from the fluid reservoir to the fluid disposed between the ultrasound element and the skin-application portion.

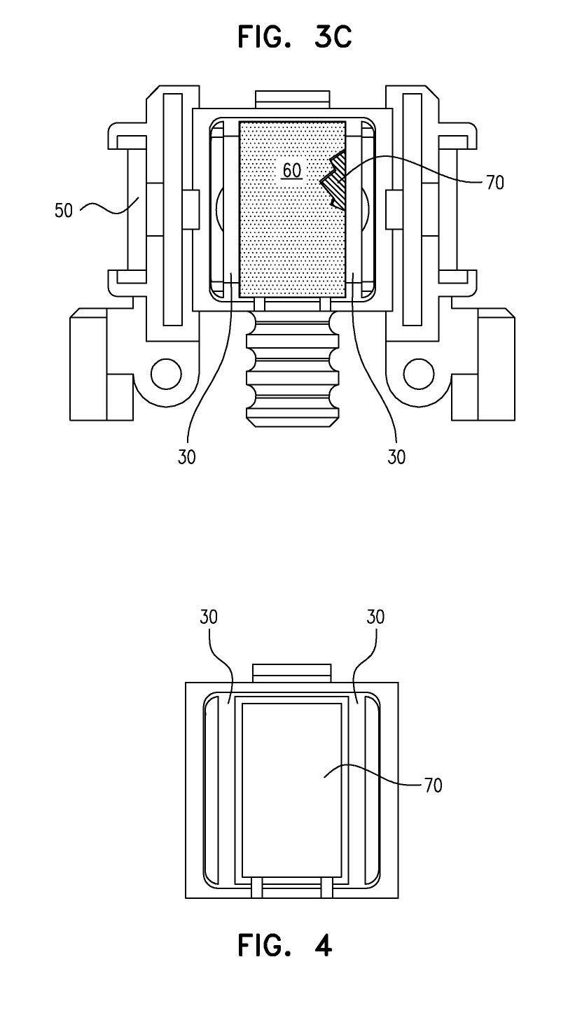

Reference is made to FIG. 4, which shows a front view of surface 70 shaped to define slots 30 and configured to be fitted into housing 50 (shown in FIGS. 2 and 3A-B). Surface 70 is typically disposed to form the outer boundary of reservoir 80 containing fluid.

Reference is again made to FIG. 1. For some applications, apparatus 20 comprises a handle 22, at a proximal portion 12 of apparatus 20, and skin-application portion 40, at a distal portion 14 of the apparatus.

Apparatus 20 comprises at least one ultrasound element 60 configured to apply ultrasound energy to the skin through skin-application portion 40. For some applications, apparatus 20 additionally comprises an extension portion 24 that extends from a distal-most tip 25 of skin-application portion 40 to a distal-most tip 27 of apparatus 20.

Extension portion 24 is typically disposed to distance skin-application portion 40 and ultrasound element 60 from a heat-sensitive portion of the subject's body, e.g., the eye, for applications in which skin-application portion 40 is moved across a face of the subject.

Extension portion 24 typically has a length of 3-10 mm, e.g., 4-mm. For some applications, a width of the extension portion is greater than a width of the skin-application portion, e.g., at least two times greater than the width of the skin-application portion. In this manner, it would be relatively difficult for a user to misuse the device and to place skin-application portion 40 against her eye, because extension portion 24 would inhibit such placement.

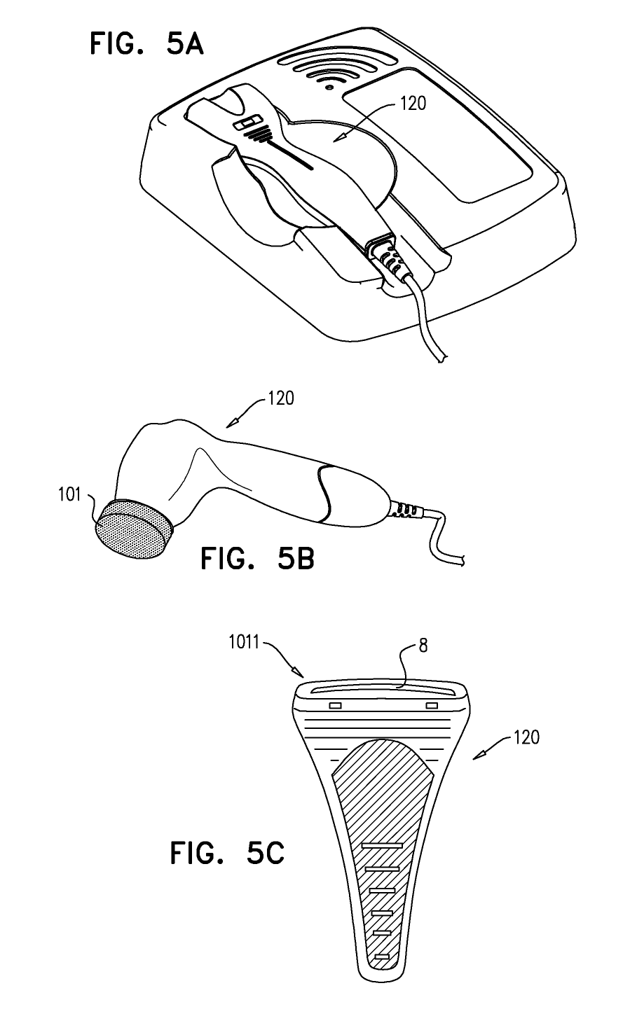

Reference is now made to FIGS. 5A-F, which are schematic illustrations of apparatus 120 for skin treatment, as provided by some applications of the present invention. Apparatus 120 is generally the same as apparatus 20 described hereinabove, except for differences specifically noted herein. Apparatus 120 comprises an ultrasound element which applies ultrasound energy, e.g., high intensity focused ultrasound (HIFU) energy, to the skin. Application of ultrasound energy to the skin surface with subsequent heating of underlying skin layers typically leads to collagen regeneration and remodeling, resulting in skin tightening and rejuvenation. It is noted that for some applications, other forms of energy may be applied, including but not limited to: laser, microwave energy, optical energy and RF waves.

Apparatus 120 is configured for use with multiple interchangeable heads configured for various functions, e.g., exfoliation and/or hair removal, and/or skin tightening treatment. For example, head 101 (shown in FIG. 5B) can be used to both perform exfoliation and to apply ultrasound energy to the skin.

Alternatively or additionally, apparatus 120 comprises head 1011 (FIG. 5C) comprising a razor 8 configured to remove hair from a skin surface of a subject (head 1011 may be used in combination with an additional razor for skin exfoliation). When using head 101 and head 1011, apparatus 120 is typically configured to transmit ultrasonic energy at a relatively low frequency e.g. 30 kHz-200 kHz. Typically, transmission of the ultrasound energy enhances the exfoliation of the skin.

A head 102 (shown in FIG. 5D) is typically used with apparatus 120 for transmission of high frequency ultrasound energy, e.g., 1 MHz-7 MHz, for enhanced cream and minerals delivery into the skin by heating of the skin. Alternatively, lower frequencies, e.g. 100 kHz-1 MHz, can be used to cause mechanical effects. For other applications, head 102 is operated for purposes of skin tightening, and rejuvenation. It is noted that for purposes of skin tightening, head 120 typically transmits ultrasound energy at high frequencies (e.g., 1 MHz-7 MHz), and at amplitudes that are generally higher than those transmitted for cream and mineral delivery.

An example of a treatment protocol using apparatus 120 involves the following steps: (a) using head 101 or 1011 to exfoliate a stratum corneum layer of skin of a subject, (b) applying cream, minerals or other cosmetic or therapeutic substances to the skin and using head 102 in order to enhance substance delivery into the skin (typically using low frequencies and amplitudes), and (c) operating head 102 at high frequencies and high amplitudes for additional effect of skin tightening and rejuvenation.

Reference is now made to FIGS. 5E-F which show apparatus 120 having combined functionalities in a single head. Apparatus 120 comprises ultrasound element 160 for transmission of ultrasound energy at high frequencies of 1 MHz-7 MHz, and an abrasive surface 165 for facilitating exfoliation.

Additional skin treatments that are provided by interchangeable heads of apparatus 120 in accordance with some applications of the present invention include hair removal, hair growth (e.g., due to improved blood circulation as a result of heating), home-treatment of sweat glands, skin treatment of larger areas such as abdomen and thighs, treatment of eye bags, and acne (it being noted that HIFU acne treatment may be combined with application of UV (ultraviolet) radiation). Interchangeable heads of apparatus 120 may also be used to provide various focal depths of treatment and/or may include phased array transducers.

For some applications, the interchangeable heads of apparatus 120 vibrate. For some applications, interchangeable heads of apparatus 120 transmit, in addition to ultrasound energy, light, laser light, RF, and/or microwave energy. Additionally or alternatively, apparatus 120 comprises an interchangeable head configured to facilitate iontophoresis.

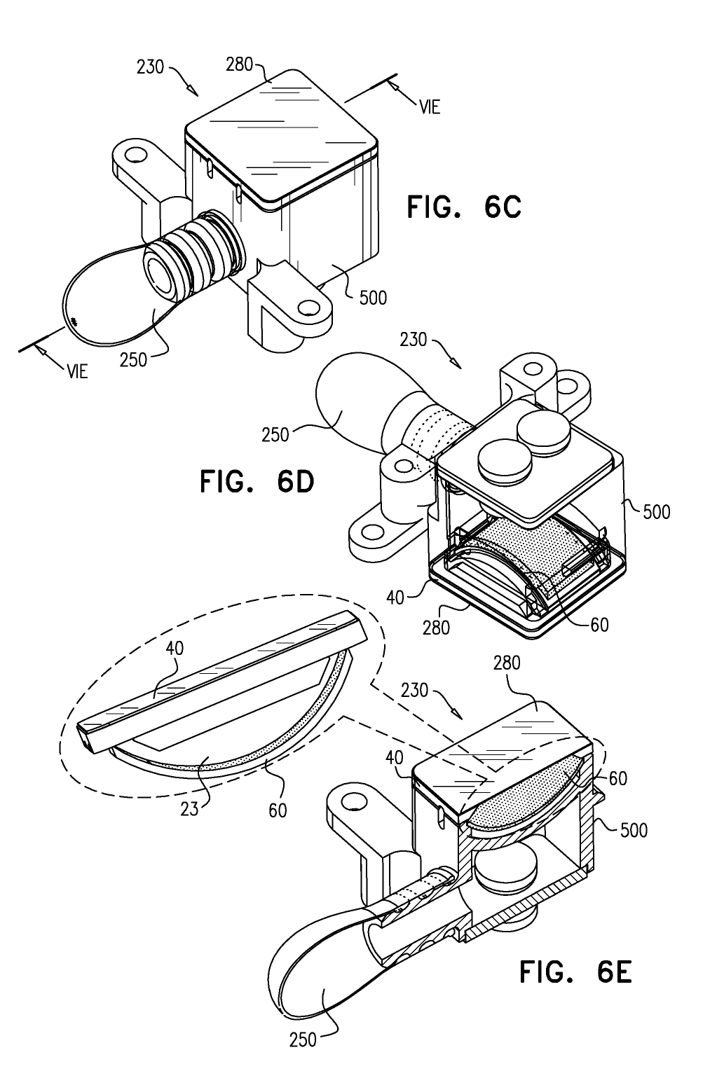

Reference is made to FIGS. 6A-E and FIG. 8. FIG. 8 is a schematic illustration of apparatus 420 for skin treatment of the subject. Apparatus 420 comprises a handle 220 and a cartridge apparatus 230 that is removably coupled to handle 220. When cartridge 230 is coupled to handle 220, apparatus 420 is activated in order to apply treatment energy to skin of the subject.

FIGS. 6A-E are schematic illustrations of components of cartridge apparatus 230 for use with a handheld device, e.g., handle 220 (shown in FIG. 8). As noted hereinabove, a user typically couples cartridge 230 to a handle to facilitate skin treatment. Cartridge 230 is removably coupled to the handle such that cartridge 230 is replaceable and may be purchased by a user separately from the handle.

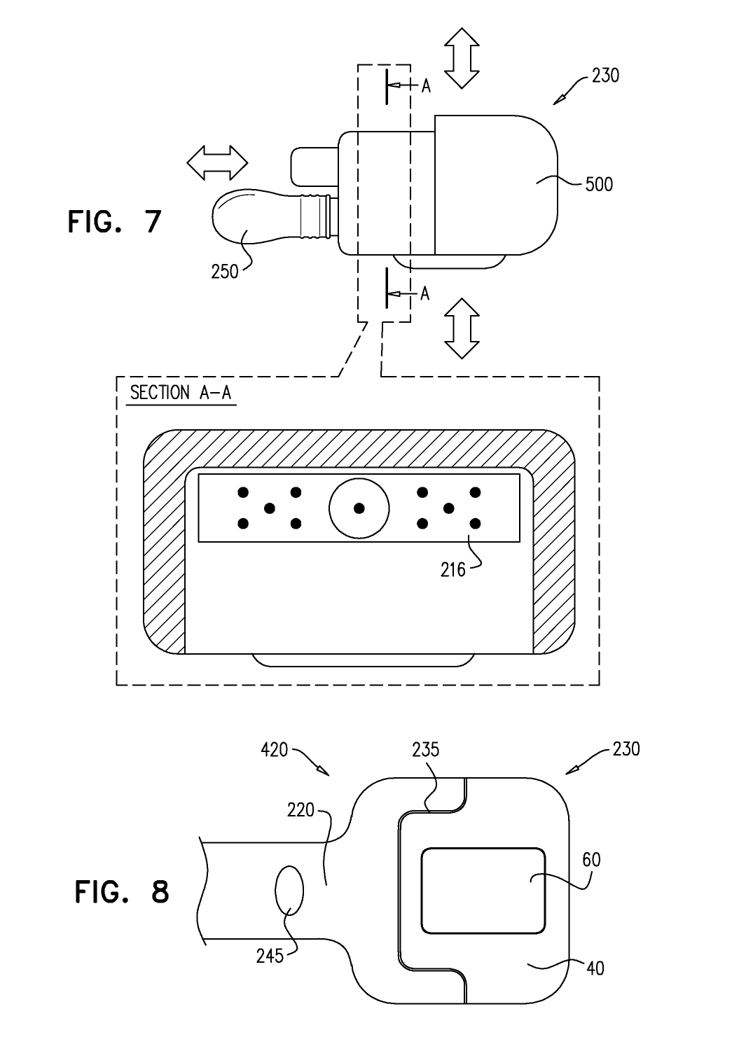

Cartridge apparatus 230 typically comprises a housing 500. Housing 500 is generally similar to housing 50 as described hereinabove, except where indicated otherwise. Housing 500 typically comprises a handle-coupling portion 235 (shown in FIG. 8) shaped and sized to securely couple housing 500 to the handle (e.g., by clicking the housing into place) and to facilitate separation of housing 500 from the handle. Typically, a user couples cartridge apparatus 230 to the handle via handle-coupling portion 235 of housing 500. Additionally, or alternatively, in order to facilitate separation of housing 500 from the handle, the handle comprises an eject button 245 (shown in FIGS. 8 and 9).

Cartridge apparatus 230 is typically shaped to define a skin-application portion 40 on an outer surface of housing 500. Skin-application portion 40 is typically positioned by a user in contact with skin of a subject and moved across the skin when cartridge apparatus 230 is attached to the handle.

Cartridge apparatus 230 additionally comprises at least one ultrasound element 60 in acoustic communication with skin-application portion 40. Ultrasound element 60 transmits ultrasound energy, e.g., high intensity focused ultrasound (HIFU) energy, to the skin through skin-application portion 40 when cartridge 230 is attached to the handle and activated to facilitate skin treatment.

Cartridge apparatus 230 further comprises an inflatable element 250 inflated with a fluid, the fluid being in fluid communication with the skin-application portion 40. Inflatable element 250 is typically composed of a compliant material which substantially does not permit leakage of fluid and exhibits high thermal stability, e.g., rubber.

For some applications, skin-application portion 40 comprises a flexible external surface, e.g., a membrane 280, which contacts the skin when skin-application portion 40 is placed on skin of the subject.

It is noted that FIG. 6A shows cartridge apparatus 230 without membrane 280 in order to present a view of ultrasound element 60. In FIG. 6D-E, both element 60 and membrane 280 are shown.

FIGS. 6B-C show membrane 280, which is disposed on the external surface of skin-application portion 40 and thus blocks the view of element 60 in these figures.

As noted hereinabove, the fluid within inflatable element 250 is in fluid communication with ultrasound element 60 and skin-application portion 40. The fluid typically applies pressure and outwardly deforms membrane 280 such that membrane 280 protrudes outwardly. Protruding of flexible surface membrane 280, results in improved contact between membrane 280 and the skin. Improved contact between membrane 280 and the skin of the subject typically enhances ultrasound treatment of the skin.

Typically, a total volume of the fluid in cartridge apparatus 230 is at least 1 cc or less than 50 cc, or between 1 and 50 cc. The inflatable element is inflated to a volume of at least 0.5 cc or less than 50 cc, or between 0.5 and 50 cc. Typically, the volume of fluid disposed between ultrasound element 60 and skin-application portion 40 is at least 0.5 cc or less than 10 cc, or between 0.5 and 10 cc.

Additionally, the inflatable element typically has a fluid pressure of at least 2 kPa or less than 25 kPa, or between 2 and 25 kPa. The volume and pressure of fluid in inflatable element 250, typically ensure a fluid volume of at least 0.5 cc or less than 10 cc, or between 0.5 and 10 cc between ultrasound element 60 and skin-application portion 40. This volume of generally facilitates inflation and protruding of membrane 280, which results in improved contact between membrane 280 and the skin.

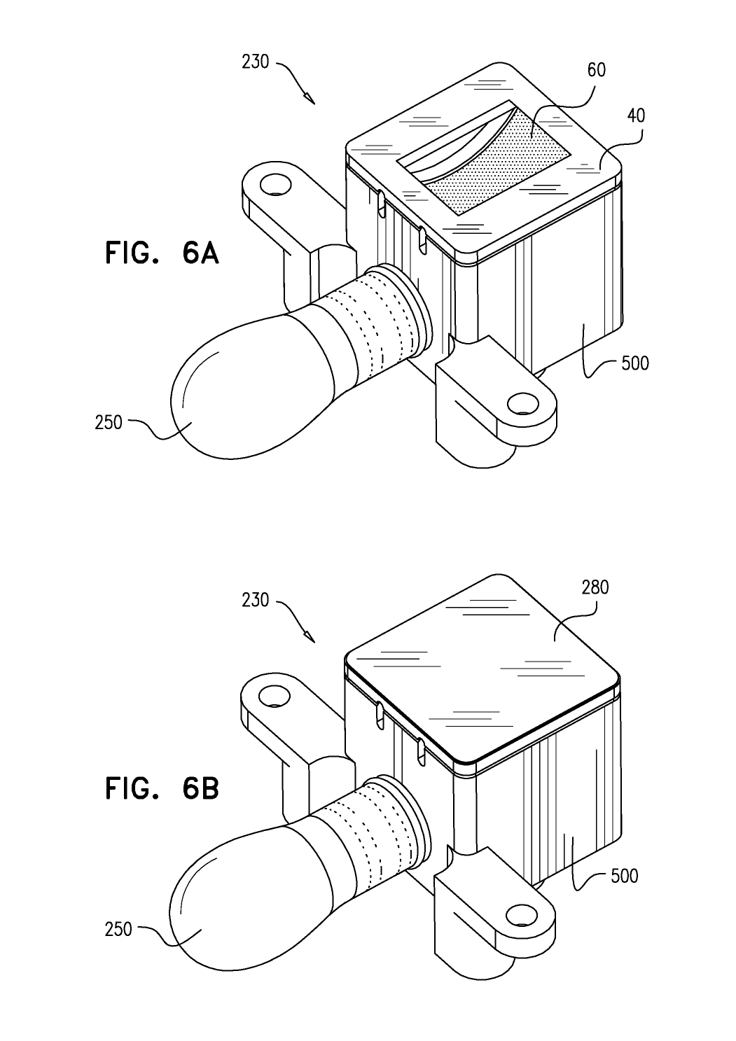

Reference is now made to FIG. 7, which is a schematic illustration of an electrical socket 216 of cartridge apparatus 230, as provided by some applications of the present invention.

Handle 220 (shown in FIG. 8), to which cartridge apparatus 230 may be removably coupled, comprises electronic circuitry which generates an electrical current when cartridge apparatus 230 is coupled to the handle. Cartridge apparatus 230 comprises electrical socket 216, which is configured to receive electrical current from the handle and convey the current to activate ultrasound element 60 to apply ultrasound energy to skin of the subject.

For some applications, cartridge apparatus 230 comprises circuitry for storing data such as calibration details of ultrasound element 60, and/or data regarding the number of uses of ultrasound element 60. For some applications, apparatus 230 is configured to block operation of ultrasound element 60 when element 60 has been used for a predetermined number of uses.

Reference is now made to FIGS. 1-3C. For some applications, apparatus 20 further comprises an inflatable element which serves generally the same function as inflatable element 250 described herein with reference to FIGS. 6A-E.

The inflatable element is typically coupled to housing 50. The inflatable element, e.g., a balloon, is inflated with a fluid, e.g. a liquid, the fluid being in fluid communication with ultrasound element 60 and skin-application portion 40.

For some applications, skin-application portion 40 of apparatus 20 comprises a flexible external surface, e.g., a membrane 280 as shown in FIGS. 6B-E. The membrane contacts the skin when the skin-application portion is placed on skin of the subject. As noted hereinabove, the fluid within the inflatable element is in fluid communication with ultrasound element 60 and skin-application portion 40. The fluid typically applies pressure and outwardly deforms the flexible surface. Inflation and protruding of the membrane by the fluid, results in improved contact between the membrane and the skin.

Typically, a total volume of the fluid in apparatus 20 is at least 1 cc or less than 50 cc or between 1 and 50 cc. The inflatable element is inflated to a volume of at least 0.5 cc or less than 50 cc, or between 0.5 and 50 cc. Typically, the volume of fluid disposed between ultrasound element 60 and skin-application portion 40 is at least 0.5 cc or less than 10 cc, or between 0.5 and 10 cc.

Additionally, the inflatable element has a fluid pressure of at least 2 kPa or less than 25 kPa, or between 2 and 25 kPa. The volume and pressure of fluid in the inflatable element typically ensure a fluid volume of at least 0.5 cc or less than 10 cc, or between 0.5 and 10 cc between the ultrasound element and the skin-application portion. This volume of at least 0.5 cc or less than 10 cc, or between 0.5 and 10 cc, generally facilitates inflation and protruding of the membrane, which results in improved contact between the membrane and the skin.

Reference is made to FIG. 9. For some applications, apparatus 20 further comprises a replaceable cartridge 231 in which the inflatable element 250 is disposed; the cartridge is removably coupled to housing 50.

The cartridge is shaped to define an outer surface. For some applications, the outer surface of the cartridge is not inflatable. For other applications, the outer surface of the cartridge inflates to a small degree.

The cartridge typically comprises a fluid port 248 (as shown in FIG. 9), which is sized and shaped to provide fluid communication between the fluid in the inflatable element and skin-application portion 40, when the cartridge is attached to housing 50.

For some applications, replaceable cartridge 231 additionally comprises an ultrasound element. For such applications, the cartridge is attached to housing 50, such that the at least one ultrasound element is placed in acoustic contact with skin of a subject and applies ultrasound energy to the skin through the skin-application portion when apparatus 20 is activated.

Reference is made to FIGS. 1-10. Apparatus 20 and apparatus 420 (comprising cartridge 230 and handle 220), are typically configured to monitor application of the energy to the skin, e.g., such that the applied energy generally does not cause undesired effects of overheating of the skin and underlying layers and does not cause heating of delicate facial structures such as an eye of the subject.

Apparatus 20 and cartridge 230 comprise skin-application portion 40, which is moved across skin of a subject for facilitating skin treatment and rejuvenation. Typically, at least one ultrasound element 60 is coupled to skin-application portion 40 and is placed in acoustic contact with the skin of the subject. Ultrasound element 60 applies ultrasound energy, e.g., high intensity focused ultrasound (HIFU) energy, to the skin. For some applications, skin-application portion 40 comprises circuitry which generates a current responsive to motion of skin-application portion 40. For some applications, motion is sensed by motion and pressure sensors 35 (shown in FIGS. 1-2); alternatively, motion is sensed by other mechanisms. A control unit receives the current and determines, in response thereto, whether the skin-application portion has been moving with respect to the skin for a predetermined duration of time. For example, the apparatus determines whether the skin-application portion has been moving for at least 0.3 seconds, e.g., for between 0.3 and 3 seconds, e.g., for more than one second. The control unit typically drives the ultrasound element to apply the ultrasound energy to the skin responsive to determining that the skin-application portion has been moving for at least 0.3 seconds with respect to the skin.

The control unit is typically configured to alter application of the energy from the ultrasound element when motion of the skin-application portion across the skin is altered. For example, application of energy from the ultrasound element may be discontinued or reduced when the skin-application portion is moving slowly or has not been moving for more than 0.3 seconds across the skin of the subject.

The inventors identified that moving of the skin-application portion across an eyelid of the subject typically does not allow for continuous movement of the skin-application portion for more than a determined time duration, e.g., for more than 0.3 seconds. Therefore, determining that the skin-application portion has not been moving for more than 0.3 seconds typically provides an indication that the skin-application portion is placed on the eyelid and the control unit discontinues application of energy to the eyelid as a safety mechanism.

As noted hereinabove, for some applications motion is sensed by motion and pressure sensors 35 (shown in FIGS. 1-2). Alternatively, motion is sensed by other sensors, e.g., a vibration sensor and/or an accelerometer sensor which measures acceleration of apparatus 20 (or apparatus 120 and 420). For example, an accelerometer is typically mounted at the head of the apparatus. When acceleration of the apparatus exceeds a threshold (e.g., due to movement of the apparatus across the skin), a “motion” state is defined, and application of ultrasound by apparatus 20 is allowed. Below this threshold, the apparatus is regarded as having no motion or too little motion, and application of ultrasound by apparatus 20 is prevented.

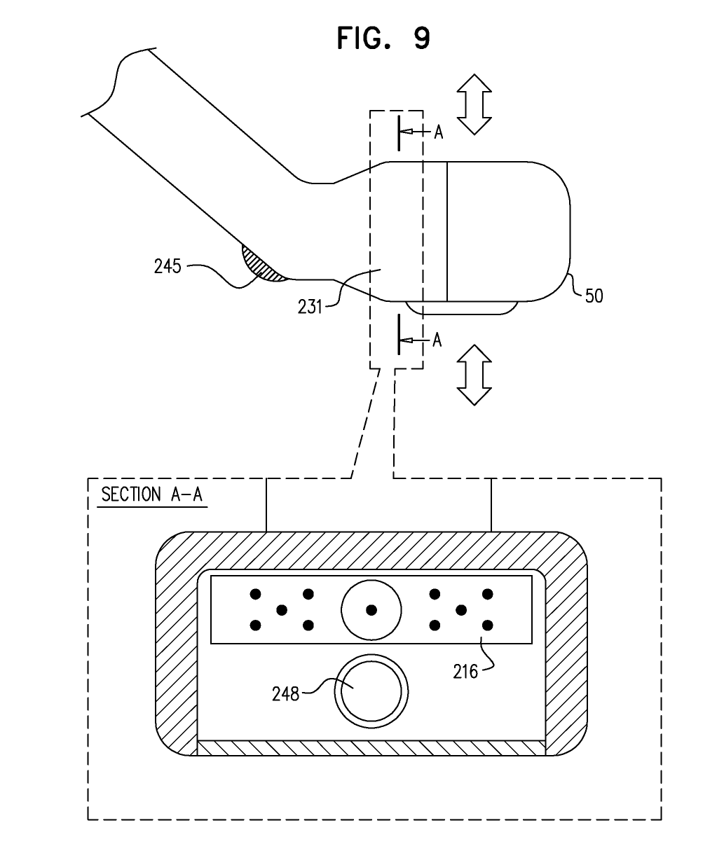

FIG. 10 is a flow chart depicting the steps for controlling application of the energy from the ultrasound element, as described hereinabove, in accordance with some applications of the present invention. FIG. 10 describes an application in which the ultrasound element is activated in response to motion of at least one second, by way of illustration and not limitation. Other durations (e.g., a duration between 0.3 seconds and one second) are also suitable, for various applications of the present invention.

For some applications, in order to enhance safety of the apparatus and prevent energy application towards the eye, the apparatus comprises one or more pressure/contact sensors 35 (shown in FIGS. 1-2). The one or more pressure sensors generate a pressure sensor signal responsive to contact of the skin-application portion with the skin. The control unit receives the pressure sensor signal and determines, responsive thereto, a degree of contact of the skin-application portion with the skin, and controls application of ultrasound energy from the ultrasound element to the skin responsive to determining the contact of the skin-application portion with the skin. For some applications, a pressure/contact sensor is placed on each side of the skin-application portion to ensure full acoustic contact of the skin-application portion with the skin surface of the subject. In particular, equal pressure sensing in both pressure sensors indicates the skin-application portion is on a flat portion of the skin (in contrast to the eye which is a curved facial structure).

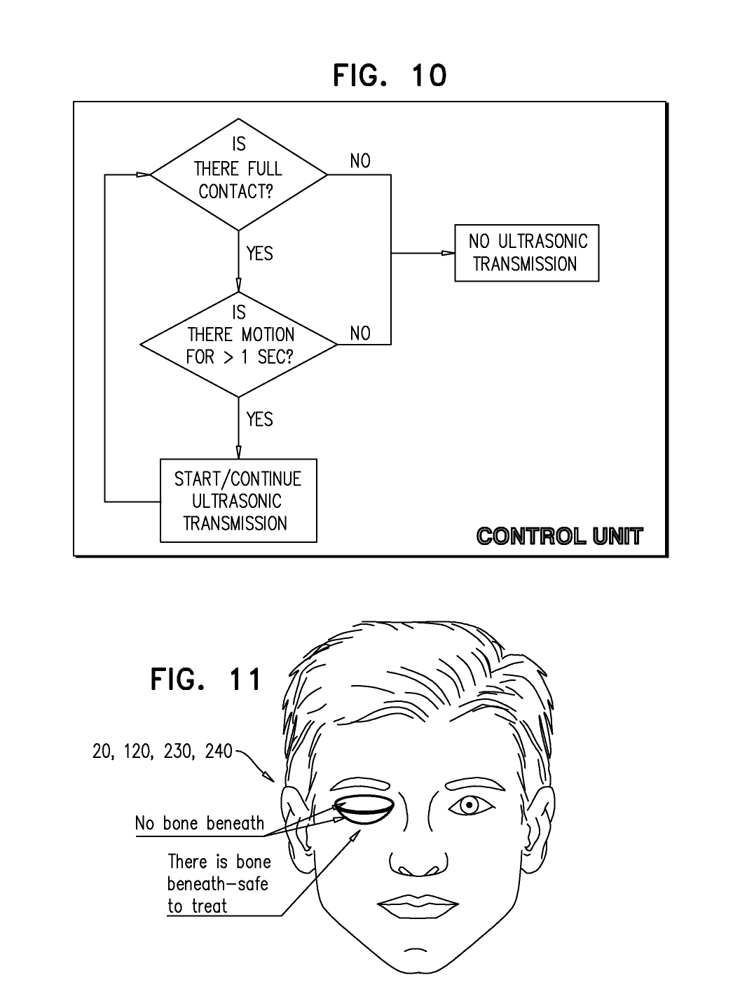

Reference is made to FIG. 11, which is a schematic illustration of a treatment site, as provided by some applications of the present invention. Additionally or alternatively to sensing physical contact of skin-application portion with the skin as an indication of sufficient acoustic contact, apparatus 20, 120, 230 and 420 are typically configured to verify sufficient acoustic contact of skin-application portion with the skin, by receiving an echo of transmitted ultrasound waves. For such applications, the ultrasound element also functions as a receiver, receiving the echo of the transmitted energy to determine whether the ultrasound energy is aimed at a bony structure underlying a skin surface (e.g., a cheek bone) or soft tissue (e.g., the eye). Typically, when the apparatus determines that the ultrasound energy is being transmitted toward soft tissue, e.g., the eye, the control unit inhibits the ultrasound element from transmitting treatment energy, e.g., high intensity focused ultrasound (HIFU) energy, to the skin. It is to be noted that, any form of monitoring described herein, e.g., motion, physical contact and/or acoustic contact monitoring, or a combination thereof, may be used in accordance with applications of the present invention.

Additionally or alternatively, the apparatus comprises other sensing modalities, e.g., an optical sensor and/or a magnetic sensor and/or an acceleration sensor and/or a capacitive sensor or a combination thereof.

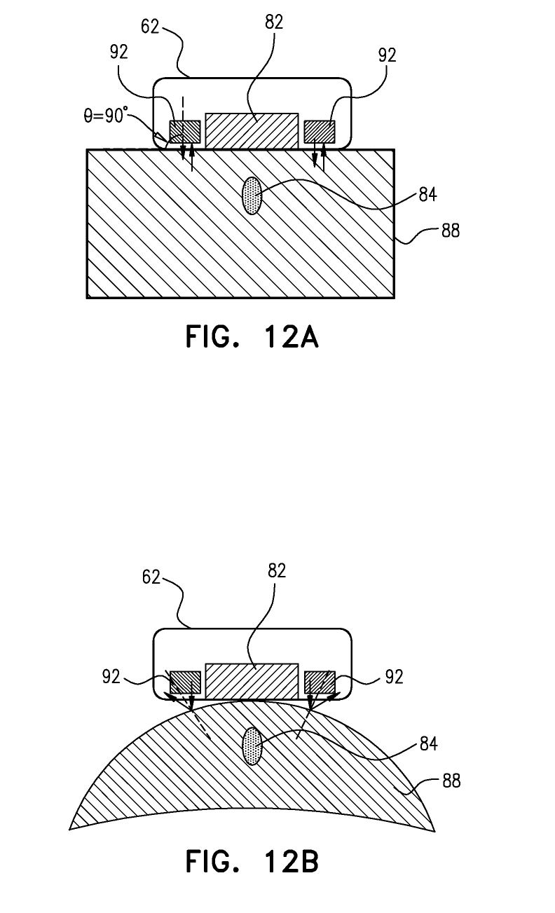

Reference is made to FIGS. 12A-D, which describe an additional or alterative mechanism for verifying that apparatus 20, 120, 230 and 420 (including ultrasound element 60, e.g., transducer 62) is placed on a generally flat surface of skin 88 (in contrast to being placed on a curved facial structure, e.g., an eye). For some applications the ultrasound transducer comprises an acoustic window 82 and one or more, typically two, optical sensors 92. The optical sensors transmit a signal towards the skin surface and receive a reflection of the energy that bounces off the skin. Typically, the optical sensors are positioned such that energy is transmitted from each sensor at a 90 degree angle (FIGS. 12A-B), or at an angle larger than 90 degrees (FIGS. 12C-D), with respect to a skin-contact surface plane of the apparatus. The signal is typically reflected back to the sensors, facilitating determination by the apparatus (e.g., by a control unit of the apparatus) of whether the transducer is placed on flat skin or curved skin (such as the eye). As shown, when the transducer is placed on a portion of flat skin, the reflected signal that reaches sensors 92 is larger (FIGS. 12A and 12C) than when the transducer is placed on a portion of curved skin (FIGS. 12B and 12D). Application of energy from the ultrasound transducer to skin 88 (e.g., toward focal volume 84) is inhibited by the control unit if it is determined that the transducer is placed on a curved skin surface such as over the eye.

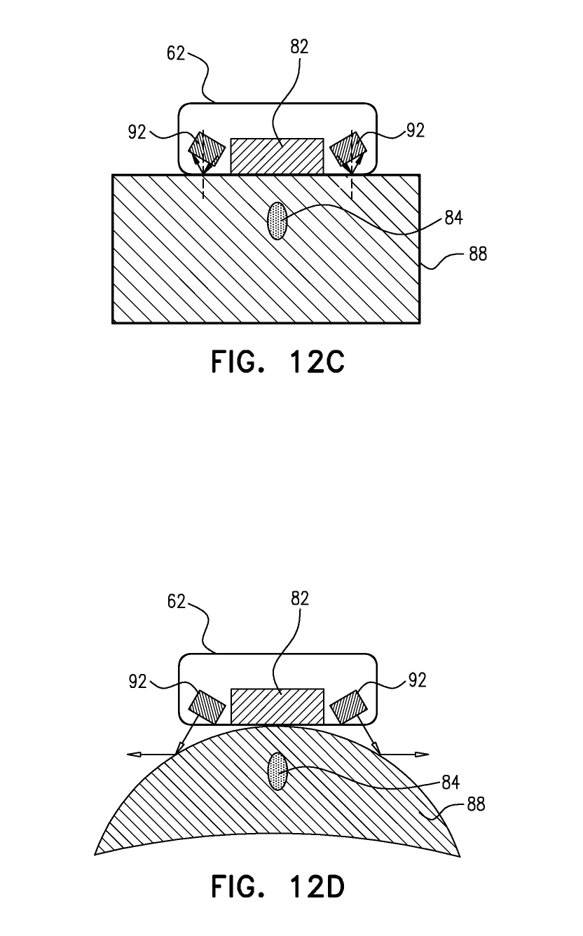

Reference is made to FIG. 13, which is a schematic illustration of apparatus 330 for treatment of eye bags. Apparatus 330 comprises an eyeglasses frame 332 and a curved ultrasound element 660 coupled to the eyeglasses frame and configured to be placed in acoustic contact with skin of an eye bag of a subject, and to apply ultrasound energy to the skin of the eye bag. (It is noted that even though lenses are shown in eyeglasses frame 332 in the figure, frame 332 may be constructed with or without lenses.) Typically, the eyeglasses frame is configured such that that the ultrasound element transmits ultrasound energy toward the bony structure beneath the eye and/or away from the eyeball, so that ultrasonic waves generally do not pass through soft tissue into the eye.

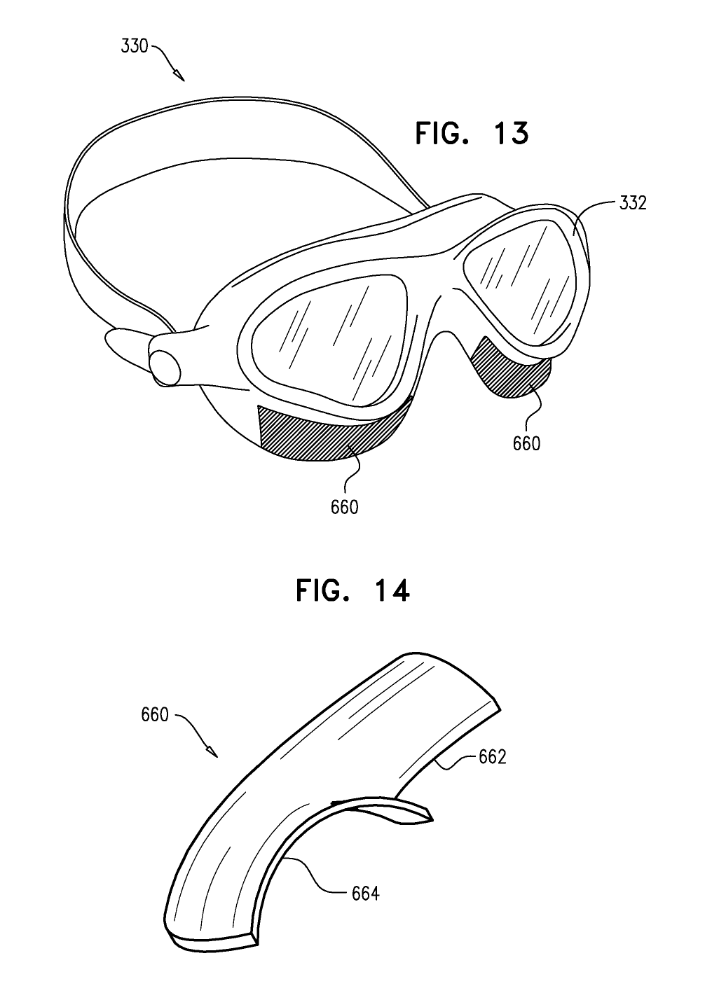

Reference is made to FIG. 14, which is a schematic illustration of curved ultrasound element 660 for use with apparatus 330 for treatment of eye bags. As shown, element 660 is shaped to define a curved ultrasound element having a coupling surface 662 and a transmitting surface 664. Coupling surface 662 typically facilitates coupling of element 660 to eyeglasses frame 332. Transmitting surface 664 typically transmits ultrasound energy, e.g., HIFU, towards skin of the subject for treatment of eye bags.

Reference is made to FIGS. 1-14. For some applications, e.g., for exfoliation treatment or treatment of sun damage, application of HIFU energy may be combined with application of Hydroquinone or TCA (Trichloroacetic Acid).

For some applications, applying HIFU energy with the use of apparatus described herein, is used to treat rosacea. HIFU treatment typically targets and damages capillaries in the dermis layer of the skin (while not harming the epidermis), causing the capillaries to be absorbed by the body's natural healing mechanism.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Cartridge apparatus for use with a handle, the apparatus comprising:
a housing, (a) comprising a handle-coupling portion shaped and sized to securely couple the housing to the handle and to facilitate separation of the housing from the handle, (b) shaped to define a skin-application portion on an outer surface of the housing, and (c) comprising at least one ultrasound element in acoustic communication with the skin-application portion and configured to transmit ultrasound energy to the skin through the skin-application portion when the cartridge is attached to the handle; and
an inflatable element inflated with a fluid, the fluid being in fluid communication with the skin-application portion.

2. The cartridge apparatus according to claim 1, wherein the skin-application portion comprises a flexible external surface configured to contact the skin, and wherein the fluid is positioned to apply pressure and outwardly deform the flexible surface.

3. The cartridge apparatus according to claim 1, wherein a volume of the fluid in fluid communication with the skin-application portion is 0.5-10 cc.

4. The cartridge apparatus according to claim 1, wherein a total volume of the fluid is 1-50 cc.

5. The cartridge apparatus according to claim 1, wherein the inflatable element has a fluid pressure of 2-25 kPa.

6. The cartridge apparatus according to claim 1, wherein a volume of the inflatable element is 0.5-50 cc.

7. The cartridge apparatus according to claim 6, wherein a pressure in the inflatable element inflated with the fluid is 2-25 kPa.

8. The cartridge apparatus according to claim 1, further comprising an electrical socket configured to receive electrical current from the handle.

9. Apparatus, comprising:
a housing;
a skin-application portion, configured to be placed in contact with skin of a subject;
at least one ultrasound element configured to be placed in acoustic contact with the skin and to apply ultrasound energy to the skin via the skin-application portion; and
an inflatable element coupled to the housing and inflated with a fluid, the fluid being in fluid communication with the ultrasound element and the skin-application portion.

10. The apparatus according to claim 9, further comprising a replaceable cartridge in which the inflatable element is disposed, and wherein the cartridge is removably coupled to the housing.

11. The apparatus according to claim 9, further comprising a replaceable cartridge in which the inflatable element and the at least one ultrasound element are disposed, and wherein the cartridge is removably coupled to the housing.

12. The apparatus according to claim 9, wherein the skin-application portion comprises a flexible external surface configured to contact the skin, and wherein the fluid applies pressure and outwardly deforms the flexible surface.

13. The apparatus according to claim 9, wherein a volume of the fluid disposed between the ultrasound element and the skin-application portion is 0.5-10 cc.

14. The apparatus according to claim 9, wherein a total volume of the fluid is 1-50 cc.

15. The apparatus according to claim 9, wherein the inflatable element has a fluid pressure of 2-25 kPa.

16. The apparatus according to claim 9, wherein the inflatable element is inflated to a volume of 0.5-50 cc.

17. The apparatus according to claim 16, wherein a pressure in the inflatable element inflated with the fluid is 2-25 kPa.

18. Cartridge apparatus, for use with an ultrasound device that includes a housing, a skin-application portion, and at least one ultrasound element, the cartridge apparatus comprising:
a cartridge configured to be removably attached to the housing;
a fluid port; and
an inflatable element disposed within the cartridge and inflated with a fluid, the fluid port being sized and shaped to provide fluid communication between the fluid in the inflatable element and the skin-application portion, when the cartridge is attached to the housing.

19. The apparatus according to claim 18, wherein the cartridge is shaped to define an outer surface thereof, and wherein the outer surface of the cartridge is not inflatable.

20. The apparatus according to claim 18, wherein the skin-application portion comprises a flexible external surface configured to contact the skin, and wherein the fluid applies pressure and outwardly deforms the flexible surface, when the cartridge is attached to the housing.

21. The apparatus according to claim 18, wherein a volume of the fluid disposed between the ultrasound element and the skin-application portion is 0.5-10 cc.

22. The apparatus according to claim 18, wherein a total volume of the fluid in the apparatus is 1-50 cc, when the cartridge is attached to the housing.

23. The apparatus according to claim 18, wherein the inflatable element has a fluid pressure of 2-25 kPa.

24. The apparatus according to claim 18, wherein the inflatable element is inflated to a volume of 0.5-50 cc.

25. The apparatus according to claim 24, wherein a pressure in the inflatable element inflated with the fluid is 2-25 kPa.

26. The apparatus according to claim 18, wherein the cartridge apparatus further comprises the at least one ultrasound element.

27. The apparatus according to claim 18, further comprising a coupling mechanism configured to couple the cartridge to the housing and to allow fluid communication between the fluid in the inflatable element and the skin-application portion, when the cartridge is attached to the housing.

28. The apparatus according to claim 27, wherein the cartridge apparatus further comprises the at least one ultrasound element.

29-47. (canceled)
